(19) United States
(12) Patent Application Publication
(10) Pub. No.: US 2010/0022944 A1
(43) Pub. Date: Jan. 28, 2010

(54) METHOD AND APPARATUS FOR TREATING AN ACUTE MYOCARDIAL INFARCTION

(76) Inventor: ROBERT L. WILCOX, BOTHELL, WA (US)

Correspondence Address: KNOBBE MARTENS OLSON & BEAR LLP, 2040 MAIN STREET, FOURTEENTH FLOOR, IRVINE, CA, 92614, US

(21) Appl. No.: 12/506,620

(22) Filed: Jul. 21, 2009

Related U.S. Application Data
(60) Provisional application No. 61/082,443, filed on Jul. 21, 2008.

(51) Int. Cl. A61M 5/00; A61N 7/00

(52) U.S. Cl. 604/22; 601/2

(57) ABSTRACT

A method and apparatus for treating an acute myocardial infarction is provided. The apparatus comprises an ultrasound catheter that can be inserted into the esophagus to deliver ultrasound energy to the heart and coronary vasculature. The ultrasound energy can enhance the effect of an intravenously administered thrombolytic drug on a blood clot within the coronary vasculature. The ultrasound catheter can have a means for promoting acoustic coupling with the esophagus and a means for preventing or reducing aspiration of fluids into the lungs.

### PRIORITY APPLICATION

This application claims the priority benefit of U.S. Provisional Application No. 61/082,443 filed Jul. 21, 2008, the entire disclosure of which is hereby incorporated by reference herein.

### BACKGROUND OF THE INVENTION

1. Field of the Invention

The present invention relates generally to the treatment of acute myocardial infarctions, and more specifically, to the treatment of acute myocardial infarctions using an ultrasound catheter.

2. Description of the Related Art

Acute myocardial infarction, also commonly known as a heart attack, occurs when blood flow to the heart is blocked or interrupted, thereby causing heart muscle damage from lack of oxygen. Most acute myocardial infarctions are caused by a blood clot that blocks one of the coronary arteries that supply blood to heart.

In some cases of acute myocardial infarction where an ECG indicates ST segment elevation, the patient is usually treated with thrombolytic therapy or percutaneous coronary intervention. The goal of both thrombolytic therapy and percutaneous coronary intervention is to remove the blood clot and restore blood flow to the heart as quickly as possible. By restoring blood flow to the heart quickly, it is possible to prevent or reduce permanent damage to the myocardium, thereby reducing mortality and morbidity, improving the patient's quality of life, and reducing the burden on the health care system. Where thrombolytic therapy is indicated, ultrasound treatment can enhance lysis of the blood clot.

Approximately 1.2 million patients in the United States are treated at a hospital each year for a heart attack. Roughly 1 out of every 5 deaths in the United States is the result of a heart attack. Accordingly, it would be desirable to provide a method and apparatus for treating an acute myocardial infarction that reduces the time required to remove the blood clot and restore blood flow to the heart.

### SUMMARY OF THE INVENTION

One embodiment of the invention comprises a method for treating a patient having an acute myocardial infarction. The method comprises providing an ultrasound catheter which comprises an elongate body having a proximal portion and a distal portion, the distal portion comprising an ultrasound energy delivery section. The method further comprises inserting the distal portion of the ultrasound catheter into the patient's esophagus and generating an ultrasonic energy field that encompasses at least a portion of the patient's heart and coronary vasculature. A thrombolytic drug can also be intravenously introduced to the patient.

In one embodiment, the ultrasound catheter further comprises a balloon mounted over the energy delivery section. The balloon can acoustically couple the ultrasound catheter to the esophagus. The balloon can be inflated with an acoustic gel or water until the balloon forms a seal around the patient's esophagus, thereby acoustically coupling the ultrasound catheter to the esophagus and reducing aspiration of fluid regurgitated from the esophagus, for example. In some embodiments, the balloon can be mounted eccentrically over the energy delivery section. Fluids in the esophagus can be aspirated via an aspiration port located in the distal portion of the ultrasound catheter.

In some embodiments, the proximal portion comprises markings that indicate the proper axial and rotational orientation of the ultrasound catheter within the patient's esophagus. The ultrasound catheter can be inserted into the patient's esophagus via the patient's mouth until the markings are aligned with the patient's mouth. In some embodiments, the ultrasound catheter can be inserted into the patient's esophagus via the patient's nose until the markings are aligned with the patient's nose. A local anesthetic lubricant can be applied to the patient's mouth and throat and/or the patient's nasal cavity and throat.

In one embodiment, an ultrasound catheter for treating a patient having an acute myocardial infarction is provided. The ultrasound catheter comprises an elongate body having a proximal portion and a distal portion and a diameter less than the patient's esophagus. The ultrasound catheter further comprises an ultrasound energy delivery section located in the distal portion, and at least one marking on the proximal portion, the marking indicating the proper axial and rotational orientation of the ultrasound catheter within the patient's esophagus. A balloon can be mounted over the energy delivery section. The balloon can be mounted eccentrically over the energy delivery section. The distal portion of the ultrasound catheter can further comprise an aspiration port and a fluid delivery port.

In some embodiments, the ultrasound catheter further comprises a cavity formed within the energy delivery section that transmits ultrasound energy poorly. The cavity can be a vacuum or can be filled with air or any other medium that transmits ultrasound energy relatively poorly.

Further embodiments of the invention are defined by the dependent claims. These and other embodiments of the present invention will become readily apparent to those skilled in the art from the following detailed description of the preferred embodiments having reference to the attached FIGS., the invention not being limited to any particular preferred embodiment(s) disclosed.

### BRIEF DESCRIPTION OF THE DRAWINGS

Exemplary embodiments of the cavitation promoting systems and methods disclosed herein are illustrated in the accompanying drawings, which are for illustrative purposes only. The drawings comprise the following figures, in which like numerals indicate like parts.

FIG. 1 is a schematic illustration of certain features of an example ultrasonic catheter.

FIG. 2 is a cross-sectional view of the ultrasonic catheter of FIG. 1 taken along line 2-2.

FIG. 3 is a schematic illustration of an elongate inner core configured to be positioned within the central lumen of the catheter illustrated in FIG. 2.

FIG. 4 is a cross-sectional view of the elongate inner core of FIG. 3 taken along line 4-4.

FIG. 5 is a schematic wiring diagram illustrating a preferred technique for electrically connecting five groups of ultrasound radiating members to form an ultrasound assembly.

FIG. 6 is a schematic wiring diagram illustrating a preferred technique for electrically connecting one of the groups of FIG. 5.

FIG. 7A is a schematic illustration of the ultrasound assembly of FIG. 5 housed within the inner core of FIG. 4.

FIG. 7B is a cross-sectional view of the ultrasound assembly of FIG. 7A taken along line 7B-7B.

FIG. 7C is a cross-sectional view of the ultrasound assembly of FIG. 7A taken along line 7C-7C.

FIG. 7D is a side view of an ultrasound assembly center wire twisted into a helical configuration.

FIG. 8 illustrates the energy delivery section of the inner core of FIG. 4 positioned within the energy delivery section of the tubular body of FIG. 2.

FIG. 9 illustrates a wiring diagram for connecting a plurality of temperature sensors with a common wire.

FIG. 10 is a block diagram of a feedback control system for use with an ultrasonic catheter.

FIG. 11 is a longitudinal cross-sectional view of selected components of an exemplary ultrasound catheter assembly that is particularly well-suited for treatment of cerebral vascular occlusions, and that includes a cavitation promoting surface.

FIG. 12 schematically illustrates an example ultrasonic energy pulse profile.

FIG. 13 is a chart showing the lysis enhancement factor of a variety of ultrasonic protocols.

FIG. 14 is a schematic illustration of an ultrasound catheter inserted into a patient's esophagus to delivery ultrasound energy to the heart.

FIG. 15 is a schematic illustration of the position of the heart and coronary vasculature with respect to the lungs.

FIGS. 16A-C show embodiments of an ultrasound catheter with a balloon for occluding the esophagus.

FIG. 17 shows a cross-section of an embodiment of an ultrasound catheter configured to generate a directional ultrasonic energy field.

FIG. 18 shows an embodiment of an ultrasound catheter with markings for orienting and positioning the ultrasound catheter within the patient.

FIGS. 19A-B show embodiments of an ultrasound catheter with means for providing acoustic coupling with the esophagus wall.

### DETAILED DESCRIPTION OF THE PREFERRED EMBODIMENT

As used herein, the term “ultrasonic energy” is used broadly, includes its ordinary meaning, and further includes mechanical energy transferred through pressure or compression waves with a frequency greater than about 20 kHz. Ultrasonic energy waves have a frequency between about 500 kHz and about 20 MHz in one example embodiment, between about 1 MHz and about 3 MHz in another example embodiment, of about 3 MHz in another example embodiment, and of about 2 MHz in another example embodiment. As used herein, the term “catheter” is used broadly, includes its ordinary meaning, and further includes an elongate flexible tube configured to be inserted into the body of a patient, such as into a body part, cavity, duct or vessel. As used herein, the term “therapeutic compound” is used broadly, includes its ordinary meaning, and encompasses drugs, medicaments, dissolution compounds, genetic materials, and other substances capable of effecting physiological functions. A mixture comprising such substances is encompassed within this definition of “therapeutic compound”. As used herein, the term “end” is used broadly, includes its ordinary meaning, and further encompasses a region generally, such that “proximal end” includes “proximal region”, and “distal end” includes “distal region”.

As expounded herein, ultrasonic energy is often used to enhance the delivery and/or effect of a therapeutic compound. For example, in the context of treating vascular occlusions, ultrasonic energy has been shown to increase enzyme mediated thrombolysis by enhancing the delivery of thrombolytic agents into a thrombus, where such agents lyse the thrombus by degrading the fibrin that forms the thrombus. The thrombolytic activity of the agent is enhanced in the presence of ultrasonic energy in the thrombus. However, it should be appreciated that the invention should not be limited to the mechanism by which the ultrasound enhances treatment unless otherwise stated. In other applications, ultrasonic energy has also been shown to enhance transfection of gene-based drugs into cells, and augment transfer of chemotherapeutic drugs into tumor cells. Ultrasonic energy delivered from within a patient's body has been found to be capable of producing non-thermal effects that increase biological tissue permeability to therapeutic compounds by up to or greater than an order of magnitude.

Use of an ultrasound catheter to deliver ultrasonic energy and a therapeutic compound directly to the treatment site mediates or overcomes many of the disadvantages associated with systemic drug delivery, such as low efficiency, high therapeutic compound use rates, and significant side effects caused by high doses. Local therapeutic compound delivery has been found to be particularly advantageous in the context of thrombolytic therapy, chemotherapy, radiation therapy, and gene therapy, as well as in applications calling for the delivery of proteins and/or therapeutic humanized antibodies. However, it should be appreciated that in certain arrangements the ultrasound catheter can also be used in combination with systemic drug delivery instead or in addition to local drug deliver. In addition, local drug delivery can be accomplished through the use of a separate device (e.g., catheter).

As will be described below, the ultrasound catheter can include one or more one or more ultrasound radiating members positioned therein. Such ultrasound radiating members can comprise a transducer (e.g., a PZT transducer), which is configured to convert electrically energy into ultrasonic energy. In such embodiments, the PZT transducer is excited by specific electrical parameters (herein “power parameters” that cause it to vibrate in a way that generates ultrasonic energy). In some embodiments, by non-linearly (e.g., randomly or pseudo randomly) varying one or more of the power parameters the effectiveness of the ultrasound catheter (e.g., the effectiveness of enhancing the removal of a thrombus) can be significantly enhanced. In addition, varying the electrical parameters may also be used in combination with varying the frequency, e.g., in a manner taught by U.S. Pat. No. 5,720,710, which is hereby incorporated by reference in its entirety.

The techniques disclosed herein are compatible with a wide variety of ultrasound catheters, several examples of which are disclosed in U.S. Pat. No. 7,220,239 (discloses catheters especially well-suited for use in the peripheral vasculature) and USA Patent Application Publication 2005/0215942 A1 (published 29 Sep. 2005; discloses catheters especially well-suited for use in the cerebral vasculature), both of which are hereby incorporated by reference in their entireties. Certain of the techniques disclosed herein are compatible with ultrasound catheters that would be unable to generate cavitation at an intravascular treatment site but for the use of such techniques.

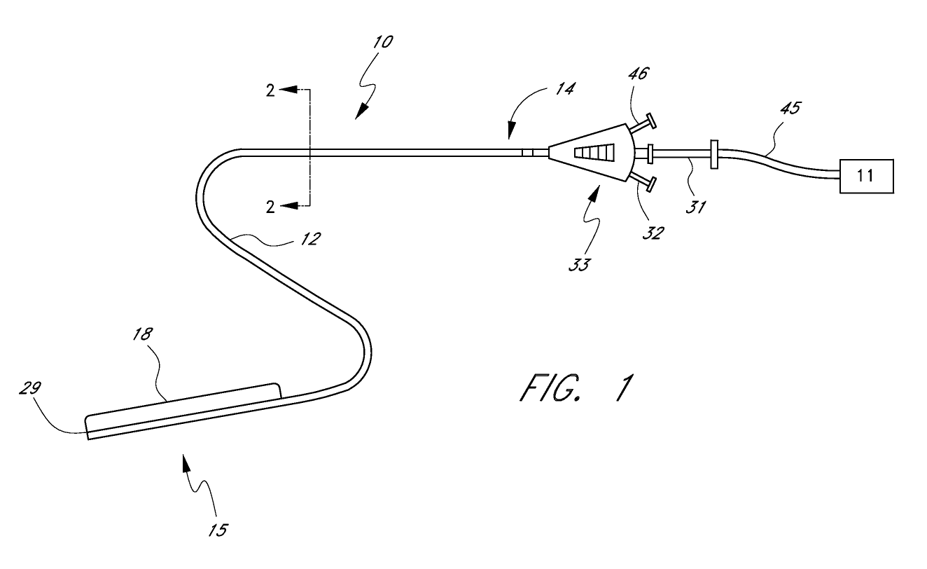

With reference to the illustrated embodiments, FIG. 1 illustrates an ultrasonic catheter 10 configured for use in a patient's vasculature or for use in the patient's esophagus or other body cavity or lumen. For example, in certain applications the ultrasonic catheter 10 is used to treat long segment peripheral arterial occlusions, such as those in the vascular system of the leg, while in other applications the ultrasonic catheter 10 is used to treat occlusions in the small vessels of the neurovasculature or other portions of the body (e.g., other distal portions of the vascular system). In other embodiments, the catheter 10 is configured for insertion into the patient's esophagus to threat a myocardial infarction. Thus, the dimensions of the catheter 10 are adjusted based on the particular application for which the catheter 10 is to be used.

The ultrasonic catheter 10 generally comprises a multi-component, elongate flexible tubular body 12 having a proximal region 14 and a distal region 15. The tubular body 12 includes a flexible energy delivery section 18 located in the distal region 15 of the catheter 10. The tubular body 12 and other components of the catheter 10 are manufactured in accordance with a variety of techniques. Suitable materials and dimensions are selected based on the natural and anatomical dimensions of the treatment site and on the desired percutaneous access site.

For example, in a preferred embodiment the proximal region 14 of the tubular body 12 comprises a material that has sufficient flexibility, kink resistance, rigidity and structural support to push the energy delivery section 18 through the patient's vasculature to a treatment site. Examples of such materials include, but are not limited to, extruded polytetrafluoroethylene (“PTFE”), polyethylenes (“PE”), polyamides and other similar materials. In certain embodiments, the proximal region 14 of the tubular body 12 is reinforced by braiding, mesh or other constructions to provide increased kink resistance and pushability. For example, in certain embodiments nickel titanium or stainless steel wires are placed along or incorporated into the tubular body 12 to reduce kinking.

The energy delivery section 18 of the tubular body 12 optionally comprises a material that (a) is thinner than the material comprising the proximal region 14 of the tubular body 12, or (b) has a greater acoustic transparency than the material comprising the proximal region 14 of the tubular body 12. Thinner materials generally have greater acoustic transparency than thicker materials. Suitable materials for the energy delivery section 18 include, but are not limited to, high or low density polyethylenes, urethanes, nylons, and the like. In certain modified embodiments, the energy delivery section 18 is formed from the same material or a material of the same thickness as the proximal region 18.

One or more fluid delivery lumens are incorporated into the tubular body 12. For example, in one embodiment a central lumen passes through the tubular body 12. The central lumen extends through the length of the tubular body 12, and is coupled to a distal exit port 29 and a proximal access port 31. The proximal access port 31 forms part of the backend hub 33, which is attached to the proximal region 14 of the catheter 10. The backend hub 33 optionally further comprises cooling fluid fitting 46, which is hydraulically connected to a lumen within the tubular body 12. The backend hub 33 also optionally comprises a therapeutic compound inlet port 32, which is hydraulically connected to a lumen within the tubular body 12. The therapeutic compound inlet port 32 is optionally also hydraulically coupled to a source of therapeutic compound via a hub such as a Luer fitting.

The catheter 10 is configured to have one or more ultrasound radiating members positioned therein. For example, in certain embodiments an ultrasound radiating member is fixed within the energy delivery section 18 of the tubular body, while in other embodiments a plurality of ultrasound radiating members are fixed to an assembly that is passed into the central lumen. In either case, the one or more ultrasound radiating members are electrically coupled to a control system 11 via cable 45. In one embodiment, the outer surface of the energy delivery 18 section can include a cavitation promoting surface configured to enhance/promote cavitation at the treatment site.

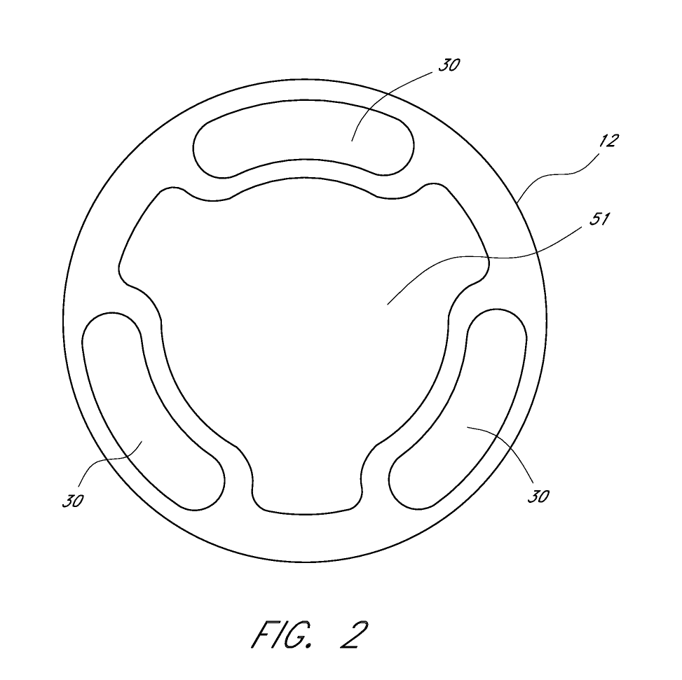

With reference to FIGS. 2-10, an exemplary arrangement of the energy delivery section 18 and other portions of the catheter 10 described above. This arrangement is particularly well-suited for treatment of peripheral vascular occlusions.

FIG. 2 illustrates a cross section of the tubular body 12 taken along line 2-2 in FIG. 1. In the embodiment illustrated in FIG. 2, three fluid delivery lumens 30 are incorporated into the tubular body 12. In other embodiments, more or fewer fluid delivery lumens can be incorporated into the tubular body 12. The arrangement of the fluid delivery lumens 30 preferably provides a hollow central lumen 51 passing through the tubular body 12. The cross-section of the tubular body 12, as illustrated in FIG. 2, is preferably substantially constant along the length of the catheter 10. Thus, in such embodiments, substantially the same cross-section is present in both the proximal region 14 and the distal region 15 of the catheter 10, including the energy delivery section 18.

In certain embodiments, the central lumen 51 has a minimum diameter greater than about 0.030 inches. In another embodiment, the central lumen 51 has a minimum diameter greater than about 0.037 inches. In one preferred embodiment, the fluid delivery lumens 30 have dimensions of about 0.026 inches wide by about 0.0075 inches high, although other dimensions may be used in other applications.

As described above, the central lumen 51 preferably extends through the length of the tubular body 12. As illustrated in FIG. 1, the central lumen 51 preferably has a distal exit port 29 and a proximal access port 31. The proximal access port 31 forms part of the backend hub 33, which is attached to the proximal region 14 of the catheter 10. The backend hub preferably further comprises cooling fluid fitting 46, which is hydraulically connected to the central lumen 51. The backend hub 33 also preferably comprises a therapeutic compound inlet port 32, which is in hydraulic connection with the fluid delivery lumens 30, and which can be hydraulically coupled to a source of therapeutic compound via a hub such as a Luer fitting.

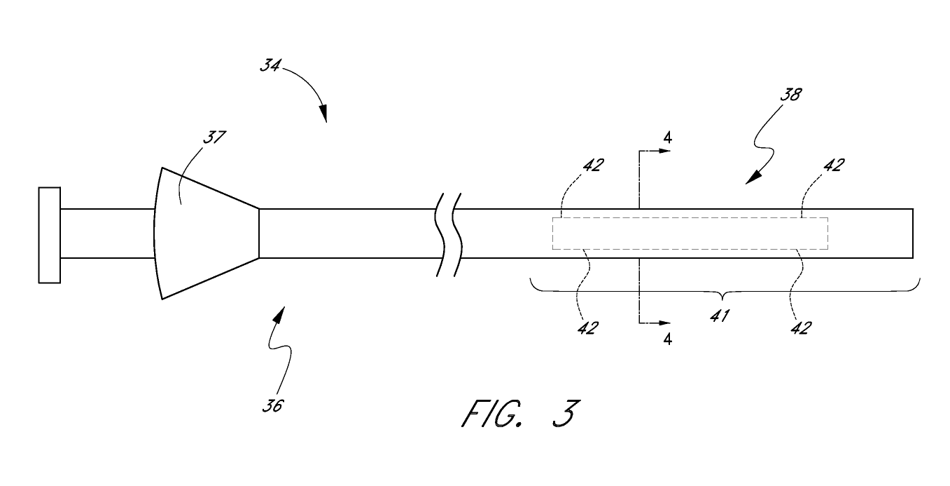

The central lumen 51 is configured to receive an elongate inner core 34 of which a preferred embodiment is illustrated in FIG. 3. The elongate inner core 34 preferably comprises a proximal region 36 and a distal region 38. Proximal hub 37 is fitted on the inner core 34 at one end of the proximal region 36. One or more ultrasound radiating members are positioned within an inner core energy delivery section 41 located within the distal region 38. The ultrasound radiating members 40 form an ultrasound assembly 42, which will be described in detail below.

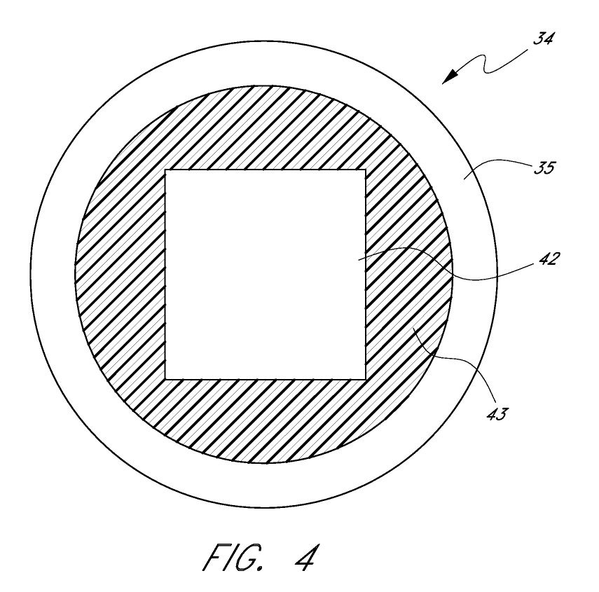

As shown in the cross-section illustrated in FIG. 4, which is taken along lines 4-4 in FIG. 3, the inner core 34 preferably has a cylindrical shape, with an outer diameter that permits the inner core 34 to be inserted into the central lumen 51 of the tubular body 12 via the proximal access port 31. Suitable outer diameters of the inner core 34 include, but are not limited to, about 0.010 inches to about 0.100 inches. In another embodiment, the outer diameter of the inner core 34 is between about 0.020 inches and about 0.080 inches. In yet another embodiment, the inner core 34 has an outer diameter of about 0.035 inches.

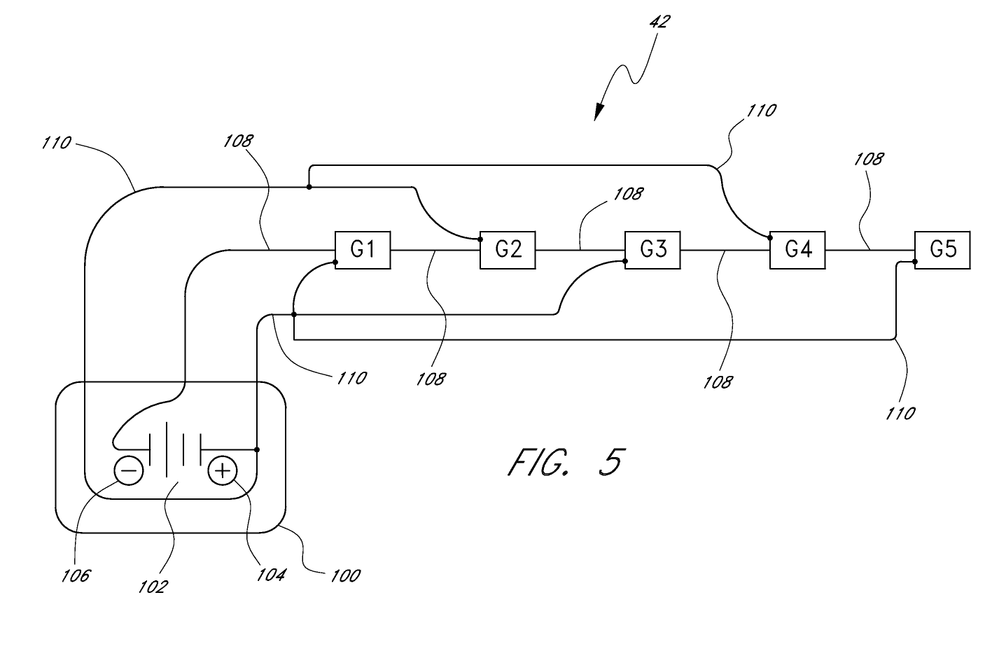

Still referring to FIG. 4, the inner core 34 preferably comprises a cylindrical outer body 35 that houses the ultrasound assembly 42. The ultrasound assembly 42 comprises wiring and ultrasound radiating members, described in greater detail in FIGS. 5 through 7D, such that the ultrasound assembly 42 is capable of radiating ultrasonic energy from the energy delivery section 41 of the inner core 34. The ultrasound assembly 42 is electrically connected to the backend hub 33, where the inner core 34 can be connected to a control system 11 via cable 45 (illustrated in FIG. 1). Preferably, an electrically insulating potting material 43 fills the inner core 34, surrounding the ultrasound assembly 42, thus preventing movement of the ultrasound assembly 42 with respect to the outer body 35. In one embodiment, the thickness of the outer body 35 is between about 0.0002 inches and 0.010 inches. In another embodiment, the thickness of the outer body 35 is between about 0.0002 inches and 0.005 inches. In yet another embodiment, the thickness of the outer body 35 is about 0.0005 inches.

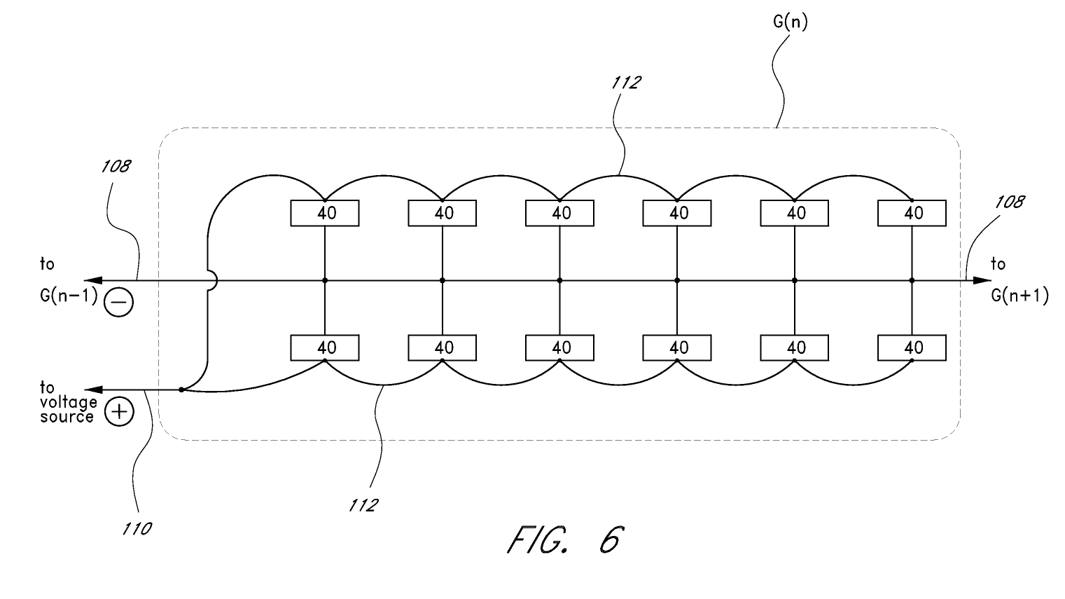

In a preferred embodiment, the ultrasound assembly 42 comprises a plurality of ultrasound radiating members 40 that are divided into one or more groups. For example, FIGS. 5 and 6 are schematic wiring diagrams illustrating one technique for connecting five groups of ultrasound radiating members 40 to form the ultrasound assembly 42. As illustrated in FIG. 5, the ultrasound assembly 42 comprises five groups G1, G2, G3, G4, G5 of ultrasound radiating members 40 that are electrically connected to each other. The five groups are also electrically connected to the control system 11.

As used herein, the terms “ultrasonic energy”, “ultrasound” and “ultrasonic” are broad terms, having their ordinary meanings, and further refer to, without limitation, mechanical energy transferred through longitudinal pressure or compression waves. Ultrasonic energy can be emitted as continuous or pulsed waves, depending on the requirements of a particular application. Additionally, ultrasonic energy can be emitted in waveforms having various shapes, such as sinusoidal waves, triangle waves, square waves, or other wave forms. Ultrasonic energy includes sound waves. In certain embodiments, the ultrasonic energy has a frequency between about 20 kHz and about 20 MHz. For example, in one embodiment, the waves have a frequency between about 500 kHz and about 20 MHz. In another embodiment, the waves have a frequency between about 1 MHz and about 3 MHz. In yet another embodiment, the waves have a frequency of about 2 MHz. The average acoustic power is between about 0.01 watts and 300 watts. In one embodiment, the average acoustic power is about 15 watts.

As used herein, the term “ultrasound radiating member” refers to any apparatus capable of producing ultrasonic energy. For example, in one embodiment, an ultrasound radiating member comprises an ultrasonic transducer, which converts electrical energy into ultrasonic energy. A suitable example of an ultrasonic transducer for generating ultrasonic energy from electrical energy includes, but is not limited to, piezoelectric ceramic oscillators. Piezoelectric ceramics typically comprise a crystalline material, such as quartz, that change shape when an electrical current is applied to the material. This change in shape, made oscillatory by an oscillating driving signal, creates ultrasonic sound waves. In other embodiments, ultrasonic energy can be generated by an ultrasonic transducer that is remote from the ultrasound radiating member, and the ultrasonic energy can be transmitted, via, for example, a wire that is coupled to the ultrasound radiating member.

Still referring to FIG. 5, the control circuitry 100 preferably comprises, among other things, a voltage source 102. The voltage source 102 comprises a positive terminal 104 and a negative terminal 106. The negative terminal 106 is connected to common wire 108, which connects the five groups G1-G5 of ultrasound radiating members 40 in series. The positive terminal 104 is connected to a plurality of lead wires 110, which each connect to one of the five groups G1-G5 of ultrasound radiating members 40. Thus, under this configuration, each of the five groups G1-G5, one of which is illustrated in FIG. 6, is connected to the positive terminal 104 via one of the lead wires 110, and to the negative terminal 106 via the common wire 108. The control circuitry can be configured as part of the control system 11 and can include circuits, control routines, controllers etc configured to vary one or more power parameters used to drive ultrasound radiating members 40,

Referring now to FIG. 6, each group G1-G5 comprises a plurality of ultrasound radiating members 40. Each of the ultrasound radiating members 40 is electrically connected to the common wire 108 and to the lead wire 110 via one of two positive contact wires 112. Thus, when wired as illustrated, a constant voltage difference will be applied to each ultrasound radiating member 40 in the group. Although the group illustrated in FIG. 6 comprises twelve ultrasound radiating members 40, one of ordinary skill in the art will recognize that more or fewer ultrasound radiating members 40 can be included in the group. Likewise, more or fewer than five groups can be included within the ultrasound assembly 42 illustrated in FIG. 5.

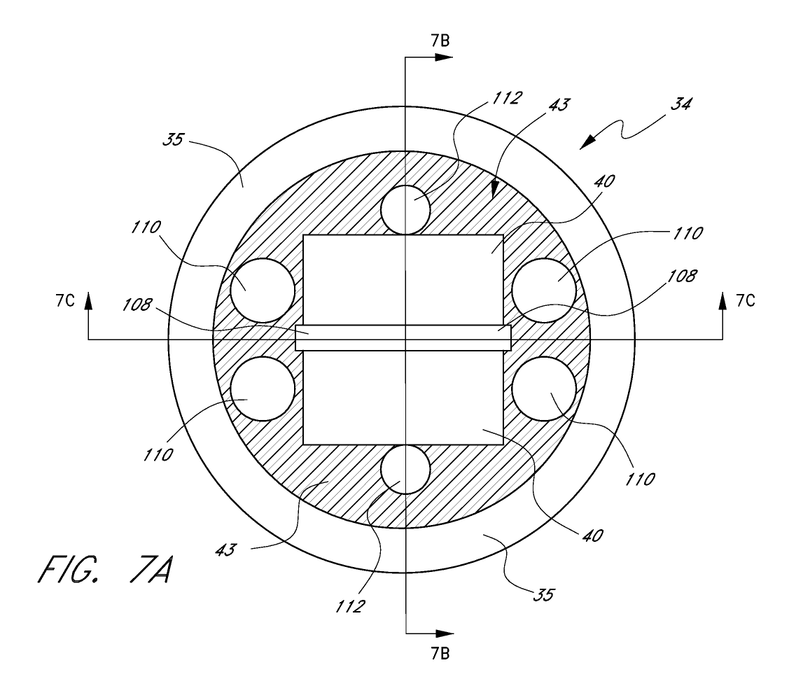

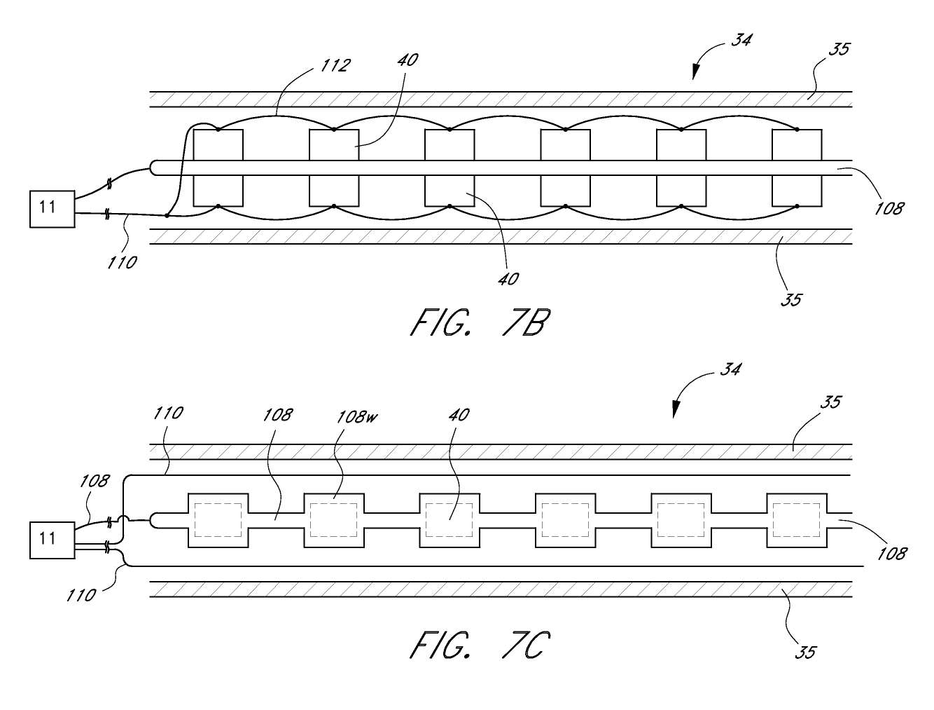

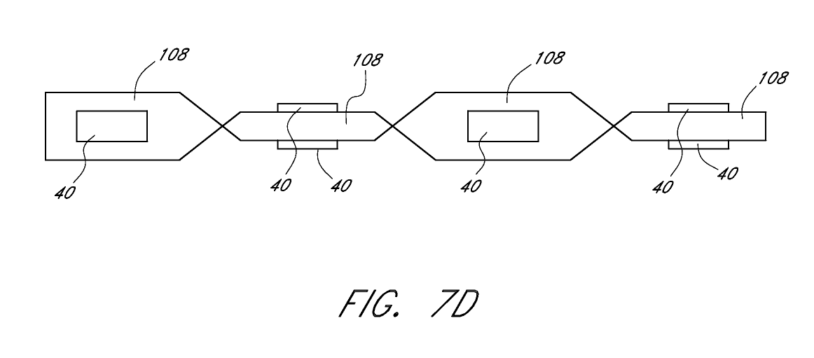

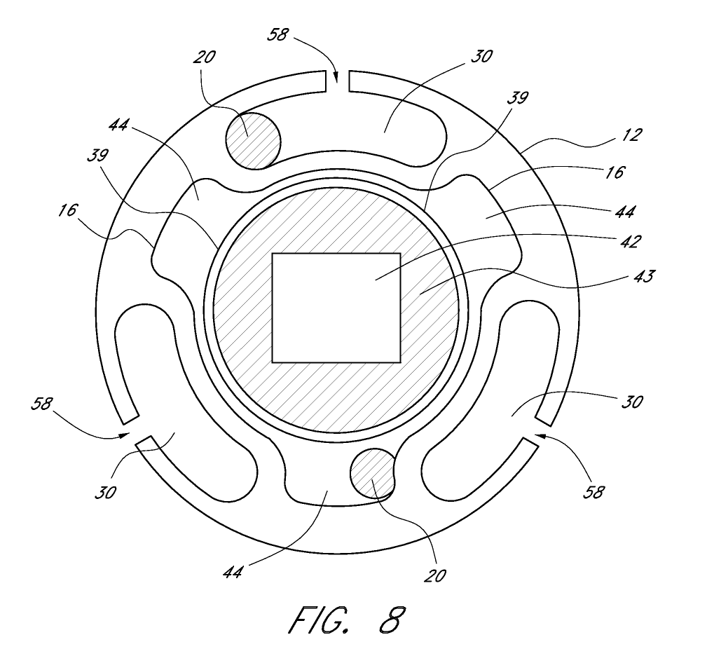

FIG. 7A illustrates one preferred technique for arranging the components of the ultrasound assembly 42 (as schematically illustrated in FIG. 5) into the inner core 34 (as schematically illustrated in FIG. 4). FIG. 7A is a cross-sectional view of the ultrasound assembly 42 taken within group G1 in FIG. 5, as indicated by the presence of four lead wires 110. For example, if a cross-sectional view of the ultrasound assembly 42 was taken within group G4 in FIG. 5, only one lead wire 110 would be present (that is, the one lead wire connecting group G5).

Referring still to FIG. 7A, the common wire 108 comprises an elongate, flat piece of electrically conductive material in electrical contact with a pair of ultrasound radiating members 40. Each of the ultrasound radiating members 40 is also in electrical contact with a positive contact wire 112. Because the common wire 108 is connected to the negative terminal 106, and the positive contact wire 112 is connected to the positive terminal 104, a voltage difference can be created across each ultrasound radiating member 40. Lead wires 110 are preferably separated from the other components of the ultrasound assembly 42, thus preventing interference with the operation of the ultrasound radiating members 40 as described above. For example, in one preferred embodiment, the inner core 34 is filled with an insulating potting material 43, thus deterring unwanted electrical contact between the various components of the ultrasound assembly 42.

FIGS. 7B and 7C illustrate cross sectional views of the inner core 34 of FIG. 7A taken along lines 7B-7B and 7C-7C, respectively. As illustrated in FIG. 7B, the ultrasound radiating members 40 are mounted in pairs along the common wire 108. The ultrasound radiating members 40 are connected by positive contact wires 112, such that substantially the same voltage is applied to each ultrasound radiating member 40. As illustrated in FIG. 7C, the common wire 108 preferably comprises wide regions 108W upon which the ultrasound radiating members 40 can be mounted, thus reducing the likelihood that the paired ultrasound radiating members 40 will short together. In certain embodiments, outside the wide regions 108W, the common wire 108 may have a more conventional, rounded wire shape.

In a modified embodiment, such as illustrated in FIG. 7D, the common wire 108 is twisted to form a helical shape before being fixed within the inner core 34. In such embodiments, the ultrasound radiating members 40 are oriented in a plurality of radial directions, thus enhancing the radial uniformity of the resulting ultrasonic energy field.

One of ordinary skill in the art will recognize that the wiring arrangement described above can be modified to allow each group G1, G2, G3, G4, G5 to be independently powered. Specifically, by providing a separate power source within the control system 11 for each group, each group can be individually turned on or off, or can be driven with an individualized power. This provides the advantage of allowing the delivery of ultrasonic energy to be “turned off” in regions of the treatment site where treatment is complete, thus preventing deleterious or unnecessary ultrasonic energy to be applied to the patient.

The embodiments described above, and illustrated in FIGS. 5 through 7, illustrate a plurality of ultrasound radiating members grouped spatially. That is, in such embodiments, all of the ultrasound radiating members within a certain group are positioned adjacent to each other, such that when a single group is activated, ultrasonic energy is delivered at a specific length of the ultrasound assembly. However, in modified embodiments, the ultrasound radiating members of a certain group may be spaced apart from each other, such that the ultrasound radiating members within a certain group are not positioned adjacent to each other. In such embodiments, when a single group is activated, ultrasonic energy can be delivered from a larger, spaced apart portion of the energy delivery section. Such modified embodiments may be advantageous in applications wherein it is desired to deliver a less focused, more diffuse ultrasonic energy field to the treatment site.

In a preferred embodiment, the ultrasound radiating members 40 comprise rectangular lead zirconate titanate (“PZT”) ultrasound transducers that have dimensions of about 0.017 inches by about 0.010 inches by about 0.080 inches. In other embodiments, other configuration may be used. For example, disc-shaped ultrasound radiating members 40 can be used in other embodiments. In a preferred embodiment, the common wire 108 comprises copper, and is about 0.005 inches thick, although other electrically conductive materials and other dimensions can be used in other embodiments. Lead wires 110 are preferably 36 gauge electrical conductors, while positive contact wires 112 are preferably 42 gauge electrical conductors. However, one of ordinary skill in the art will recognize that other wire gauges can be used in other embodiments.

As described above, suitable frequencies for the ultrasound radiating member 40 include, but are not limited to, from about 20 kHz to about 20 MHz. In one embodiment, the frequency is between about 500 kHz and 20 MHz, and in another embodiment 1 MHz and 3 MHz. In yet another embodiment, the ultrasound radiating members 40 are operated with a frequency of about 2 MHz.

FIG. 8 illustrates the inner core 34 positioned within the tubular body 12. Details of the ultrasound assembly 42, provided in FIG. 7A, are omitted for clarity. As described above, the inner core 34 can be slid within the central lumen 51 of the tubular body 12, thereby allowing the inner core energy delivery section 41 to be positioned within the tubular body energy delivery section 18. For example, in a preferred embodiment, the materials comprising the inner core energy delivery section 41, the tubular body energy delivery section 18, and the potting material 43 all comprise materials having a similar acoustic impedance, thereby minimizing ultrasonic energy losses across material interfaces.

FIG. 8 further illustrates placement of fluid delivery ports 58 within the tubular body energy delivery section 18. As illustrated, holes or slits are formed from the fluid delivery lumen 30 through the tubular body 12, thereby permitting fluid flow from the fluid delivery lumen 30 to the treatment site. Thus, a source of therapeutic compound coupled to the inlet port 32 provides a hydraulic pressure which drives the therapeutic compound through the fluid delivery lumens 30 and out the fluid delivery ports 58.

By evenly spacing the fluid delivery lumens 30 around the circumference of the tubular body 12, as illustrated in FIG. 8, a substantially even flow of therapeutic compound around the circumference of the tubular body 12 can be achieved. In addition, the size, location and geometry of the fluid delivery ports 58 can be selected to provide uniform fluid flow from the fluid delivery ports 30 to the treatment site. For example, in one embodiment, fluid delivery ports closer to the proximal region of the energy delivery section 18 have smaller diameters then fluid delivery closer to the distal region of the energy delivery section 18, thereby allowing uniform delivery of fluid across the entire energy delivery section.

For example, in one embodiment in which the fluid delivery ports 58 have similar sizes along the length of the tubular body 12, the fluid delivery ports 58 have a diameter between about 0.0005 inches to about 0.0050 inches. In another embodiment in which the size of the fluid delivery ports 58 changes along the length of the tubular body 12, the fluid delivery ports 58 have a diameter between about 0.001 inches to about 0.005 inches in the proximal region of the energy delivery section 18, and between about 0.005 inches to 0.0020 inches in the distal region of the energy delivery section 18. The increase in size between adjacent fluid delivery ports 58 depends on the material comprising the tubular body 12, and on the size of the fluid delivery lumen 30. The fluid delivery ports 58 can be created in the tubular body 12 by punching, drilling, burning or ablating (such as with a laser), or by any other suitable method. Therapeutic compound flow along the length of the tubular body 12 can also be increased by increasing the density of the fluid delivery ports 58 toward the distal region 15 of the tubular body 12.

It should be appreciated that it may be desirable to provide non-uniform fluid flow from the fluid delivery ports 58 to the treatment site. In such embodiment, the size, location and geometry of the fluid delivery ports 58 can be selected to provide such non-uniform fluid flow.

Referring still to FIG. 8, placement of the inner core 34 within the tubular body 12 further defines cooling fluid lumens 44. Cooling fluid lumens 44 are formed between an outer surface 39 of the inner core 34 and an inner surface 16 of the tubular body 12. In certain embodiments, a cooling fluid can is introduced through the proximal access port 31 such that cooling fluid flow is produced through cooling fluid lumens 44 and out distal exit port 29 (see FIG. 1). The cooling fluid lumens 44 are preferably evenly spaced around the circumference of the tubular body 12 (that is, at approximately 120.degree. increments for a three-lumen configuration), thereby providing uniform cooling fluid flow over the inner core 34. Such a configuration is desirably to remove unwanted thermal energy at the treatment site. As will be explained below, the flow rate of the cooling fluid and the power to the ultrasound assembly 42 can be adjusted to maintain the temp of the inner core energy delivery section 41 within a desired range.

In a preferred embodiment, the inner core 34 can be rotated or moved within the tubular body 12. Specifically, movement of the inner core 34 can be accomplished by maneuvering the proximal hub 37 while holding the backend hub 33 stationary. The inner core outer body 35 is at least partially constructed from a material that provides enough structural support to permit movement of the inner core 34 within the tubular body 12 without kinking of the tubular body 12. Additionally, the inner core outer body 35 preferably comprises a material having the ability to transmit torque. Suitable materials for the inner core outer body 35 include, but are not limited to, polyimides, polyesters, polyurethanes, thermoplastic elastomers and braided polyimides.

In a preferred embodiment, the fluid delivery lumens 30 and the cooling fluid lumens 44 are open at the distal end of the tubular body 12, thereby allowing the therapeutic compound and the cooling fluid to pass into the patient's vasculature at the distal exit port. Or, if desired, the fluid delivery lumens 30 can be selectively occluded at the distal end of the tubular body 12, thereby providing additional hydraulic pressure to drive the therapeutic compound out of the fluid delivery ports 58. In either configuration, the inner core 34 can prevented from passing through the distal exit port by making providing the inner core 34 with a length that is less than the length of the tubular body. In other embodiments, a protrusion is formed on the internal side of the tubular body in the distal region 15, thereby preventing the inner core 34 from passing through the distal exit port.

In still other embodiments, the catheter 10 further comprises an occlusion device (not shown) positioned at the distal exit port 29. The occlusion device preferably has a reduced inner diameter that can accommodate a guidewire, but that is less than the inner diameter of the central lumen 51. Thus, the inner core 34 is prevented from extending through the occlusion device and out the distal exit port 29. For example, suitable inner diameters for the occlusion device include, but are not limited to, about 0.005 inches to about 0.050 inches. In other embodiments, the occlusion device has a closed end, thus preventing cooling fluid from leaving the catheter 10, and instead recirculating to the proximal region 14 of the tubular body 12. These and other cooling fluid flow configurations permit the power provided to the ultrasound assembly 42 to be increased in proportion to the cooling fluid flow rate. Additionally, certain cooling fluid flow configurations can reduce exposure of the patient's body to cooling fluids.

In certain embodiments, as illustrated in FIG. 8, the tubular body 12 further comprises one or more temperature sensors 20, which are preferably located within the energy delivery section 18. In such embodiments, the proximal region 14 of the tubular body 12 includes a temperature sensor lead which can be incorporated into cable 45 (illustrated in FIG. 1). Suitable temperature sensors include, but are not limited to, temperature sensing diodes, thermistors, thermocouples, resistance temperature detectors (“RTDs”) and fiber optic temperature sensors which use thermalchromic liquid crystals. Suitable temperature sensor 20 geometries include, but are not limited to, a point, a patch or a stripe. The temperature sensors 20 can be positioned within one or more of the fluid delivery lumens 30 (as illustrated), and/or within one or more of the cooling fluid lumens 44.

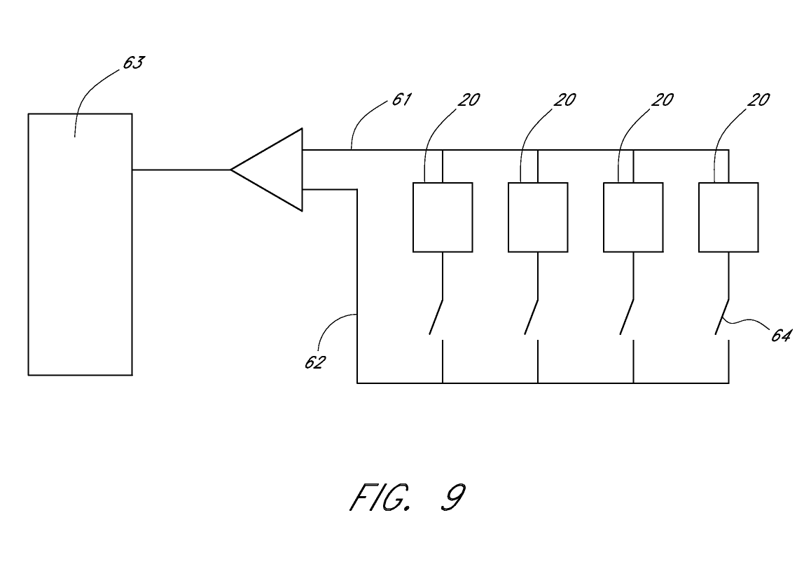

FIG. 9 illustrates one embodiment for electrically connecting the temperature sensors 20. In such embodiments, each temperature sensor 20 is coupled to a common wire 61 and is associated with an individual return wire 62. Accordingly, n+1 wires can be used to independently sense the temperature at n distinct temperature sensors 20. The temperature at a particular temperature sensor 20 can be determined by closing a switch 64 to complete a circuit between that thermocouple's individual return wire 62 and the common wire 61. In embodiments wherein the temperature sensors 20 comprise thermocouples, the temperature can be calculated from the voltage in the circuit using, for example, a sensing circuit 63, which can be located within the external control circuitry 100.

In other embodiments, each temperature sensor 20 is independently wired. In such embodiments, 2n wires through the tubular body 12 to independently sense the temperature at n independent temperature sensors 20. In still other embodiments, the flexibility of the tubular body 12 can be improved by using fiber optic based temperature sensors 20. In such embodiments, flexibility can be improved because only n fiber optic members are used to sense the temperature at n independent temperature sensors 20.

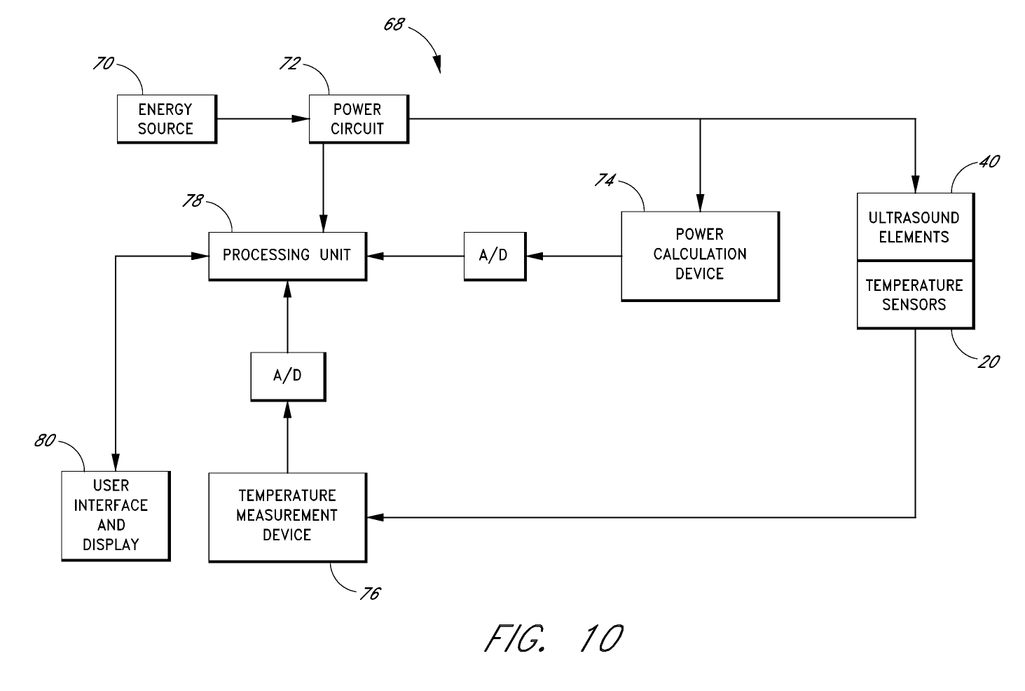

FIG. 10 illustrates one embodiment of a feedback control system 68 that can be used with the catheter 10. The feedback control system 68 can be integrated into the control system 11 that is connected to the inner core 34 via cable 45 (as illustrated in FIG. 1). The feedback control system 68 allows the temperature at each temperature sensor 20 to be monitored and allows the output power of the energy source 70 to be adjusted accordingly. A physician can, if desired, override the closed or open loop system.

The feedback control system 68 preferably comprises an energy source 70, power circuits 72 and a power calculation device 74 that is coupled to the ultrasound radiating members 40. A temperature measurement device 76 is coupled to the temperature sensors 20 in the tubular body 12. A processing unit 78 is coupled to the power calculation device 74, the power circuits 72 and a user interface and display 80.

In operation, the temperature at each temperature sensor 20 is determined by the temperature measurement device 76. The processing unit 78 receives each determined temperature from the temperature measurement device 76. The determined temperature can then be displayed to the user at the user interface and display 80.

The processing unit 78 comprises logic for generating a temperature control signal. The temperature control signal is proportional to the difference between the measured temperature and a desired temperature. The desired temperature can be determined by the user (at set at the user interface and display 80) or can be preset within the processing unit 78.

The temperature control signal is received by the power circuits 72. The power circuits 72 are preferably configured to adjust the power level, voltage, phase and/or current of the electrical energy supplied to the ultrasound radiating members 40 from the energy source 70. For example, when the temperature control signal is above a particular level, the power supplied to a particular group of ultrasound radiating members 40 is preferably reduced in response to that temperature control signal. Similarly, when the temperature control signal is below a particular level, the power supplied to a particular group of ultrasound radiating members 40 is preferably increased in response to that temperature control signal. After each power adjustment, the processing unit 78 preferably monitors the temperature sensors 20 and produces another temperature control signal which is received by the power circuits 72.

The processing unit 78 preferably further comprises safety control logic. The safety control logic detects when the temperature at a temperature sensor 20 has exceeded a safety threshold. The processing unit 78 can then provide a temperature control signal which causes the power circuits 72 to stop the delivery of energy from the energy source 70 to that particular group of ultrasound radiating members 40.

Because, in certain embodiments, the ultrasound radiating members 40 are mobile relative to the temperature sensors 20, it can be unclear which group of ultrasound radiating members 40 should have a power, voltage, phase and/or current level adjustment. Consequently, each group of ultrasound radiating member 40 can be identically adjusted in certain embodiments. In a modified embodiment, the power, voltage, phase, and/or current supplied to each group of ultrasound radiating members 40 is adjusted in response to the temperature sensor 20 which indicates the highest temperature. Making voltage, phase and/or current adjustments in response to the temperature sensed by the temperature sensor 20 indicating the highest temperature can reduce overheating of the treatment site.

The processing unit 78 also receives a power signal from a power calculation device 74. The power signal can be used to determine the power being received by each group of ultrasound radiating members 40. The determined power can then be displayed to the user on the user interface and display 80.

As described above, the feedback control system 68 can be configured to maintain tissue adjacent to the energy delivery section 18 below a desired temperature. For example, it is generally desirable to prevent tissue at a treatment site from increasing more than 6.degree. C. As described above, the ultrasound radiating members 40 can be electrically connected such that each group of ultrasound radiating members 40 generates an independent output. In certain embodiments, the output from the power circuit maintains a selected energy for each group of ultrasound radiating members 40 for a selected length of time.

The processing unit 78 can comprise a digital or analog controller, such as for example a computer with software. When the processing unit 78 is a computer it can include a central processing unit (“CPU”) coupled through a system bus. As is well known in the art, the user interface and display 80 can comprise a mouse, a keyboard, a disk drive, a display monitor, a nonvolatile memory system, or any another. Also preferably coupled to the bus is a program memory and a data memory.

In lieu of the series of power adjustments described above, a profile of the power to be delivered to each group of ultrasound radiating members 40 can be incorporated into the processing unit 78, such that a preset amount of ultrasonic energy to be delivered is pre-profiled. In such embodiments, the power delivered to each group of ultrasound radiating members 40 can then be adjusted according to the preset profiles.

The ultrasound radiating members are preferably operated in a pulsed mode. For example, in one embodiment, the time average electrical power supplied to the ultrasound radiating members is between about 0.001 watts and 5 watts and can be between about 0.05 watts and 3 watts. In certain embodiments, the time average electrical power over treatment time is approximately 0.45 watts or 1.2 watts. The duty cycle is between about 0.01% and 90% and can be between about 0.1% and 50%. In certain embodiments, the duty ratio is approximately 7.5%, 15% or a variation between 1% to 30%. The pulse averaged electrical power can be between about 0.01 watts and 20 watts and can be between approximately 0.1 watts and 20 watts. In certain embodiments, the pulse averaged electrical power is approximately 4 watts, 8 watts, 16 watts, or a variation of 1 to 8 watts. As will be described above, the amplitude, pulse width, pulse repetition frequency, average acoustic pressure or any combination of these parameters can be constant or varied during each pulse or over a set of portions. In a non-linear application of acoustic parameters the above ranges can change significantly. Accordingly, the overall time average electrical power over treatment time may stay the same but not real-time average power.

In one embodiment, the pulse repetition rate is preferably between about 1 Hz and 2 kHz and more can be between about 1 Hz and 50 Hz. In certain preferred embodiments, the pulse repetition rate is approximately 30 Hz, or a variation of 10 to 40Hz. The pulse duration or width is can be between about 0.5 millisecond and 50 milliseconds and can be between about 0.1 millisecond and 25 milliseconds. In certain embodiments, the pulse duration is approximately 2.5 milliseconds, 5 or a variation of 1 to 8 milliseconds. In addition, the average acoustic pressure can be between about 0.1 to 2 MPa or in another embodiment between about 0.5 or 0.74 to 1.7 MPa.

In one particular embodiment, the transducers are operated at an average power of approximately 0.6 watts, a duty cycle of approximately 7.5%, a pulse repetition rate of 30 Hz, a pulse average electrical power of approximately 8 watts and a pulse duration of approximately 2.5 milliseconds.

The ultrasound radiating member used with the electrical parameters described herein preferably has an acoustic efficiency than 50% and can be greater than 75%. The ultrasound radiating member can be formed a variety of shapes, such as, cylindrical (solid or hollow), flat, bar, triangular, and the like. The length of the ultrasound radiating member is preferably between about 0.1 cm and about 0.5 cm. The thickness or diameter of the ultrasound radiating members is preferably between about 0.02 cm and about 0.2 cm.

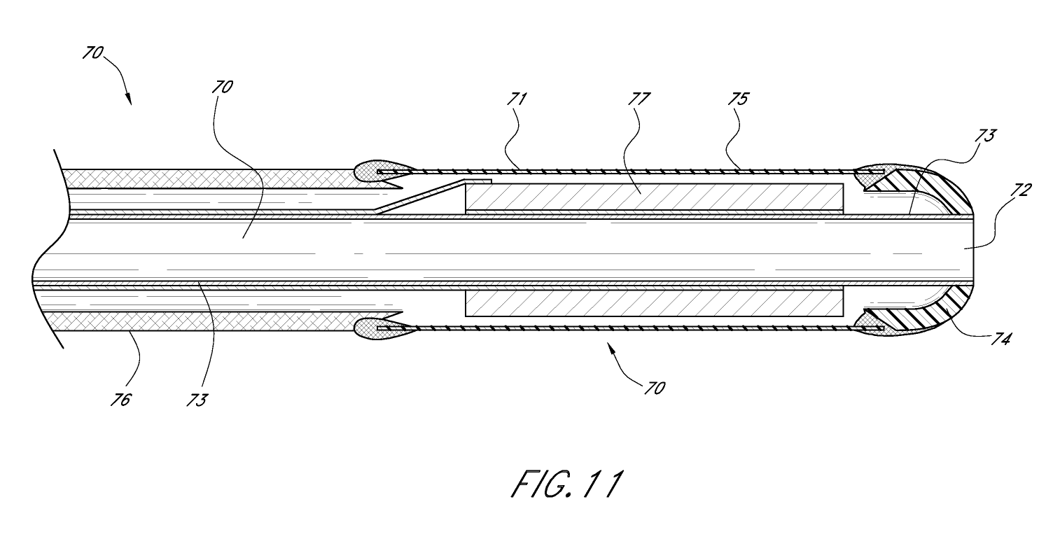

With reference now to FIG. 11, the energy delivery section of an ultrasound catheter that is configured for treating small vessels (e.g., for treatment of cerebral vascular occlusions) is shown and that includes an optional cavitation promoting surface 71. In this embodiment, the catheter includes an inner core 73 that defines a utility lumen 72 configured to pass materials such as a guidewire, a therapeutic compound and/or a cooling fluid. The catheter assembly 70 further includes a distal tip element 74 and a hollow cylindrical ultrasound radiating member 77 that is mounted on the inner core 73. Certain of these components are optional, and are omitted from alternative embodiments. In an example embodiment, the diameter of the catheter outer body 76 is less than about 5 French, although other dimensions are used in other embodiments. In addition, although only a single ultrasound element is shown, in modified embodiments, more one ultrasound element can be mounted along the lumen 72.

In example embodiments, the ultrasound radiating member 77 illustrated in FIG. 11 is a tubular piezoceramic transducer that is able to radiate ultrasonic energy in a length mode, a thickness mode, and a circumferential mode. The ultrasound radiating member 77 is capable of generating a peak acoustic pressures that are preferably between about 0.7 MPa and about 10 MPa, and that are more preferably between about 1.2 MPa and about 6 MPa. However such parameters may be different if the catheter includes cavitation promoting surfaces or other modifications.

In a modified embodiment, the ultrasound radiating member 77 has a resonant frequency greater than or equal to approximately 1 MHz in the thickness mode. In certain embodiments, the ultrasound radiating member included in an ultrasound catheter optionally includes an electrode, such as a nickel-plated electrode, that enables electrical wires to be soldered thereto.

As will be described below, the ultrasound catheter includes one or more one or more ultrasound radiating members positioned therein. Such ultrasound radiating members can comprise a transducer (e.g., a PZT transducer), which is configured to convert electrically energy into ultrasonic energy. In such embodiments, the PZT transducer is excited by specific electrical parameters (herein “power parameters” or “acoustic parameters” that cause it to vibrate in a way that generates ultrasonic energy). In some embodiments, by non-linearly varying (e.g., randomly or pseudo randomly) one or more of the power parameters the effectiveness of the ultrasound catheter (e.g., the effectiveness of enhancing the removal of a thrombus) can be significantly enhanced. By non-linearly varying one or more of the power parameters the ultrasound radiating members create nonlinear acoustic pressure, which as described above can increase the effectiveness of the acoustic pressure in enhancing a therapeutic compound. Examples of nonlinear variances include, but are not limited to, multi variable variations, variations as a function of a complex equation, sinusoidal variations, exponential variations, random variations, pseudo random variations and/or arbitrary variations.

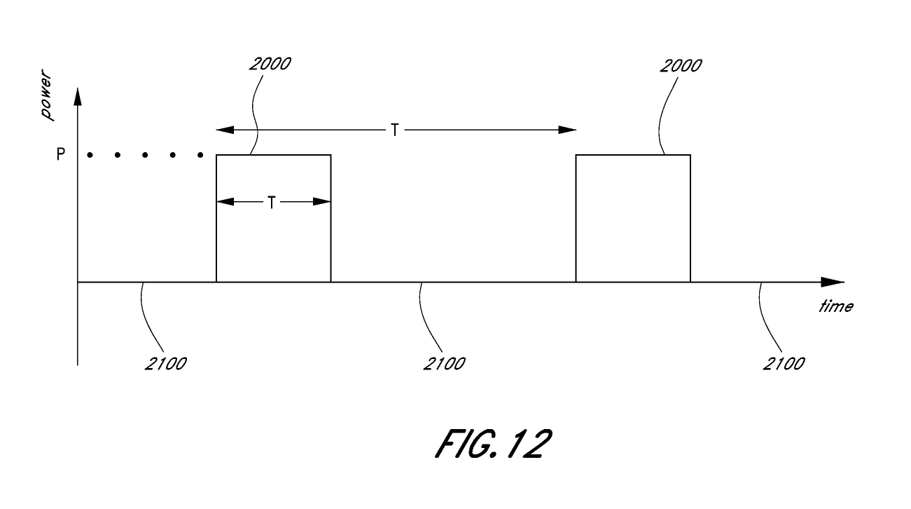

FIG. 12 illustrates certain power parameters which can used to drive the ultrasound radiating members. As shown, the members can be driven a series of pulses 2000 having peak power P or amplitude and duration τ. During these pulses 2000, the ultrasound radiating members as driven at a certain frequency f as described above by the electrical current. The pulses 2000 can be separated by “off” periods 2100. The cycle period T is defined as the time between pulse initiations, and thus the pulse repetition frequency (“PRF”) is given by T⁻¹.

Examples of non-linear variation include, but are not limited to, simple or complex variable or multi-variable equations, varying randomly, pseudo randomly and/or in an arbitrary manner. The average power of each cycle period can be adjusted by manipulating one or more parameters of the waveform in the cycle period, such as, but not limited to, peak power P, reduced power P′, pulse repetition frequency, pulse duration τ, and duty cycle.

The pulse amplitude, pulse width and pulse repetition frequency during each pulse can also be constant or varied in a non-linear fashion as described herein. Other parameters are used in other embodiments depending on the particular application.

The acoustic protocols tested are summarized in Table 1 provided below. “PW” represents pulse width and “PRF” represents pulse repetition frequency. Ranges indicate that the parameter was varied randomly within the range shown. For example, for the R3P-d protocol, peak power was varied from 1.6 to 7.9 W, pulse width was varied from 1.16 to 8.16 ms, and pulse repetition frequency was varied from 10 to 40 Hz.

TABLE 1
Description of acoustic protocols

| | Average | Peak | | | | |
|---|---|---|---|---|---|---|
| Acoustic Protocol | Power | Power | | PW | | PRF |
| Neurowave E11 | 0.45 W | 5.3 | W | 2.8 | ms | 30 Hz |
| (E11-S) | | | | | | |
| R3P-d (R1.4) | 0.45 W | 1.6-7.9 | W | 1.16-8.16 | ms | 10-40 Hz |
| R1P-f (R5.5) | 0.45 W | 3.75 | W | 0.31-19.53 | ms | 30 Hz |
| R1P-g (R5.6) | 0.90 W | 3.75 | W | 0.62-39.07 | ms | 30 Hz |
| R2P-a (R6.0) | 0.45 W | 1.6-7.9 | W | 0.54-9.8 | ms | 30 Hz |
| R2P-b (R6.1) | 0.90 W | 1.6-7.9 | W | 1.09-19.6 | ms | 30 Hz |

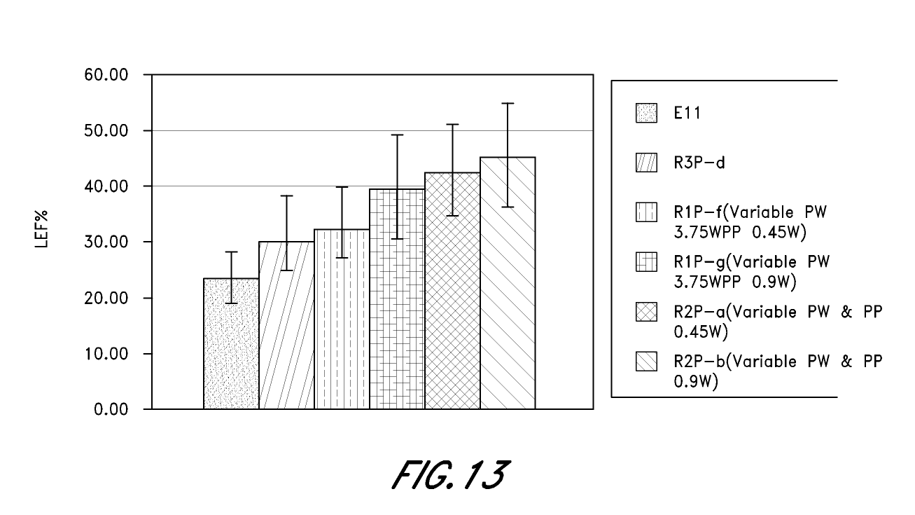

FIG. 13 shows the lysis enhancement factor (LEF %) for the protocols tested. The results indicate that varying peak power and pulse width simultaneously in the randomization protocol give significantly better lysis enhancement in the test environment than varying either parameter alone or when they are varied together with pulse repetition frequency. In addition, higher peak powers generally yielded improved lysis response. It should be appreciated that the Lysis enhancement factor is only one measure of the efficacy of the treatment and that the methods and technique described above may have additional and/or different efficacy benefits in situ.

In addition, although many embodiments have been described in the context of an intravascular catheter it should be appreciated that the non-linear application of one or more power parameters can also be applied to non-intravascular catheters or devices and/or non catheter applications. For example, the non-linear varying of one or more power parameters may also find utility in applications in which the ultrasound is applied externally (with respect to the body or with respect to the vascular system). For example, as further described below, the ultrasound catheters can be used in the treatment of acute myocardial infarction by, for example, insertion into the esophagus and delivering ultrasound energy to the heart. In addition, in some embodiments, the therapeutic affects of the ultrasound can be utilized alone without a therapeutic compound.

### Treatment of Acute Myocardial Infarction

One non-intravascular application in which the ultrasound catheter 10 can be used is in the treatment of an acute myocardial infarction, particularly in the emergency room (ER) setting. Where thrombolytic therapy is indicated, the ultrasound catheter 10 can be used to delivery ultrasound energy to the clot to enhance thrombolysis. Ultrasound energy can be delivered by the ultrasound catheter 10 before, during, and/or after administration of a thrombolytic drug to the patient. The ultrasound energy can be delivered in either a linear or non-linear manner. The thrombolytic drug can be introduced intravenously through a standard IV access. As illustrated in FIG. 14, the ultrasound catheter 10 can be inserted into the esophagus 200, where ultrasound energy can be transmitted through the esophageal wall and to the blood clot in the heart 202 and/or coronary vasculature 204. The ability to deliver ultrasound energy to substantially the entire heart 202 and coronary vasculature 204 can be important in the emergency room treatment of an acute myocardial infraction because the precise location of the blood clot within the coronary vasculature 204 may not be known for a period of time following the initial diagnosis of the infarction.

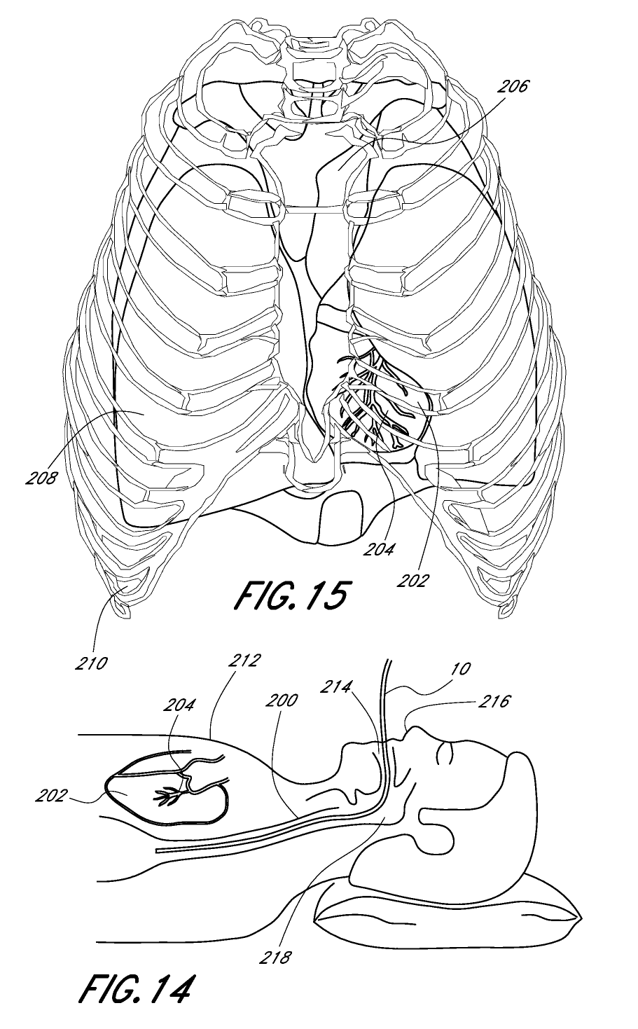

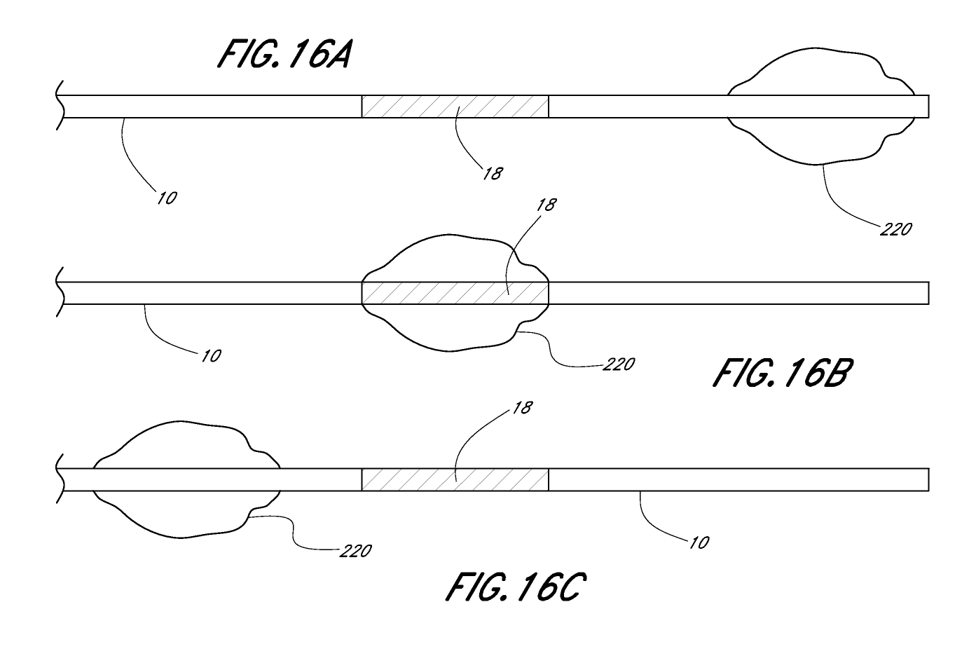

A trans-esophageal approach for the delivery of ultrasound energy is preferable in some situations over alternative approaches such as trans-thoracic or trans-abdominal access for a variety of reasons. For trans-thoracic delivery of ultrasound energy using an external ultrasound transducer placed on the exterior of the patient chest, the ultrasound energy is radiated through the patient's chest wall, sternum 206, ribs 210 and through a small window between the lungs 208 where the heart 202 and portions of the coronary vasculature 204 are exposed, as shown in FIG. 15. Because bone absorbs ultrasound energy and the air in the lungs 208 transmits ultrasound energy relatively poorly, trans-thoracic access to the heart 202 is relatively limited. In addition, because the ultrasound energy must pass through the sternum 206, ribs 210 and/or chest wall before reaching the heart 202, relatively high levels of ultrasound energy generally must be delivered for a sufficient amount of ultrasound energy to reach the exposed portions of the heart 202 and coronary vasculature 204. However, using high levels of ultrasound energy can cause burning or damage to tissue. Furthermore, because absorption of ultrasound energy is generally intensified at an air interface, nearby lung tissue interfaces are at risk of excessive heating.

Trans-abdominal access from the abdomen and through the diaphragm has three primary challenges. First, a trans-abdominal approach can be very painful for the patient because general anesthesia may not be available in an emergency room setting. The organs of the abdomen and the muscles of the thorax are sensitive to pain and would require general anesthesia to make the procedure tolerable. Second, the risk of infection in the peritoneum, and the risk of air leak in the pleural space add to the challenge of either of these punctures of the patient's abdomen and diaphragm. Finally, the underlying organs would be at risk of abrasion, nicking, or other minor injuries which may not be well managed in the emergency room setting.

In contrast, the esophageal approach to the heart 202 is a standard path for delivering ultrasound energy to the heart in a minimally invasive setting for applications such as imaging. Gastroscopy and trans-esophageal echocardiography (TEE) are commonly performed in locations without angiographic imaging capability, so this approach is appropriate for the ER. These procedures involve inserting a device into the patient's esophagus 200, and are generally tolerated by the patient despite being uncomfortable. As shown in FIGS. 14 and 15, the ultrasound energy emitted by the ultrasound emitting device is not obstructed by the patient's lungs 208, sternum 206, ribs 210 or chest wall 212, allowing ultrasound energy to be delivered to substantially the entire heart 202. Designing a catheter 10 similar to a TEE imaging device will provide familiarity to the operator, thereby reducing the level of training required to operate the catheter 10 and increasing the rate of adoption. Accordingly, a smaller catheter with a linear array of transducers can be appropriate, as described in detail above.

In some embodiments, by making the catheter 10 with a very small cross-sectional diameter, the unpleasant sensation caused by insertion and placement of the catheter 10 into the esophagus 200 can be reduced. The diameter of the catheter 10 is less than the diameter of the esophagus 200, and is some embodiments, is substantially less than the diameter of the esophagus 200. Insertion of the catheter 10 into the esophagus 200 can be accomplished via the mouth 214 or nasal cavities 216. In some embodiments, the catheter 10 can be introduced with a stylet for additional column strength. Once the stylet is removed, the catheter 10 becomes flaccid and causes less of a disturbance or irritation to the back of the throat 218. A local anesthetic lubricant can be applied to the patient's mouth 214, nasal cavities 216, throat 218 and/or the catheter 10 to reduce the gag reflex and the patient's discomfort. In addition, in some embodiments, a sedative can also be given with the local anesthesia, also to reduce the gag reflex and the patient's discomfort. Once the catheter 10 is in place, there are generally very few complaints of gagging.

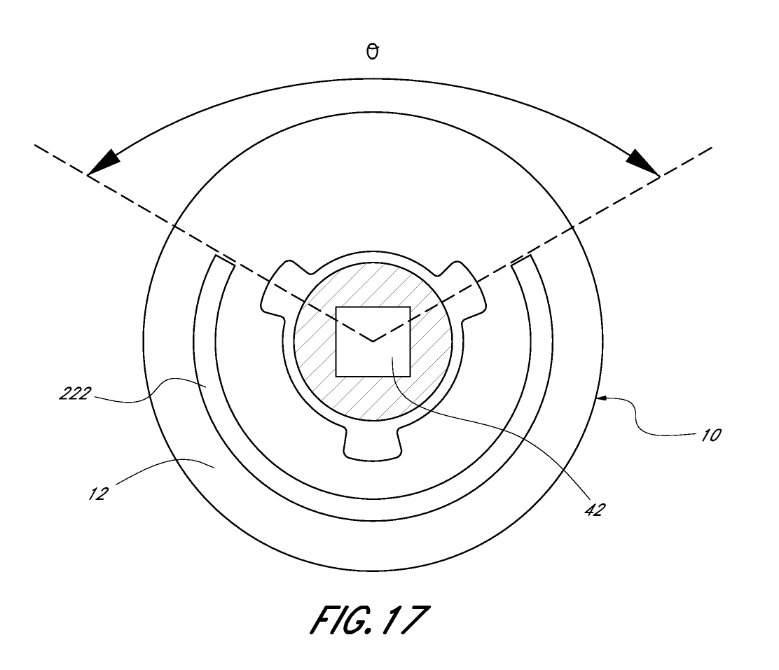

However, patients may be nauseated, which can be a symptom of myocardial infarction, and insertion of the catheter may stimulate the gag reflex, causing the patient to vomit. Therefore, in some embodiments, the catheter 10 includes means for reducing aspiration of fluids into the patient's airway passages. For example, in some embodiments as shown in FIG. 16A, a balloon 220 can be mounted at the distal end or the distal portion of the catheter 10, and inflated with an acoustic gel or water during or after catheter 10 placement to impede stomach contents from traveling up the esophagus. In other embodiments as shown in FIG. 16B, the balloon 220 can be mounted over the energy delivery section 18 and can be inflated with an acoustic gel or water. In other embodiments, the balloon 220 can be mounted proximally the energy delivery section 18.

In some embodiments as shown in FIG. 19B, the catheter 10 can have at least one aspiration port 226 and at least one aspiration lumen. In other embodiments, the catheter 10 can have a plurality of aspirations ports 226. In some embodiments, the catheter 10 can also have a plurality of aspiration lumens. The aspiration ports and lumens can be in fluid communication with a vacuum or reduced pressure source, allowing the aspiration ports and lumens to aspirate fluids in the esophagus, such as vomit. In some embodiments, the aspiration ports are located at the distal end and/or on the distal portion of the catheter 10.

In some embodiments, an aspiration catheter can be used to aspirate fluids in the esophagus. The aspiration catheter can be attached to or separate from the ultrasound catheter 10. Similarly, in some embodiments a balloon occlusion catheter can be used to occlude the esophagus. The balloon occlusion catheter can be attached to or separate from the ultrasound catheter.

Targeting the delivery of ultrasound energy can be accomplished in at least several ways. One way is to use a rotational orientation for the ultrasound transducers that generates a directional ultrasonic energy field that can be appropriate for anyone of a given body mass. For example, the catheter 10 can be constructed to emit ultrasound energy along an arc θ as illustrated in FIG. 17 which shows a cross-section of an embodiment of an ultrasound catheter 10 generating a directional ultrasonic energy field. In some embodiments, arc θ can be less than about 180 degrees, or less than about 120 degrees, or less than about 90 degrees, or less than about 60 degrees, or less than about 30 degrees. The catheter 10 can generate the directional ultrasonic energy field by a variety of means. For example, directional ultrasound transducers that preferentially emit ultrasound energy in a particular direction can be aligned to form an ultrasound assembly 42 along arc θ. In addition, ultrasound transparent or transmissive materials can be used to fabricate the portion of the sheath or tubular body 12 along arc θ while ultrasound opaque material is used to fabricate the other portions of the sheath. For example, a cavity 222 can be formed within the portion of energy delivery portion 18 to block and/or reduce ultrasound energy transmission through portions of the catheter 10. The cavity 222 can be filled with an ultrasound opaque filling, such as air, or can be evacuated to form a vacuum. A linear array of ultrasound transducers can be used to generate an ultrasonic energy field of a particular length.

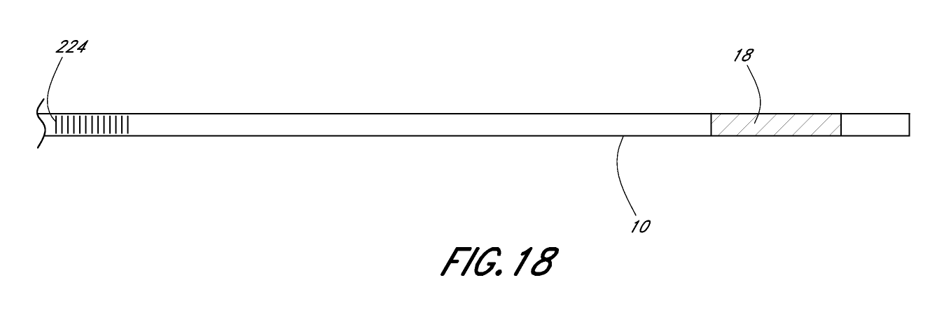

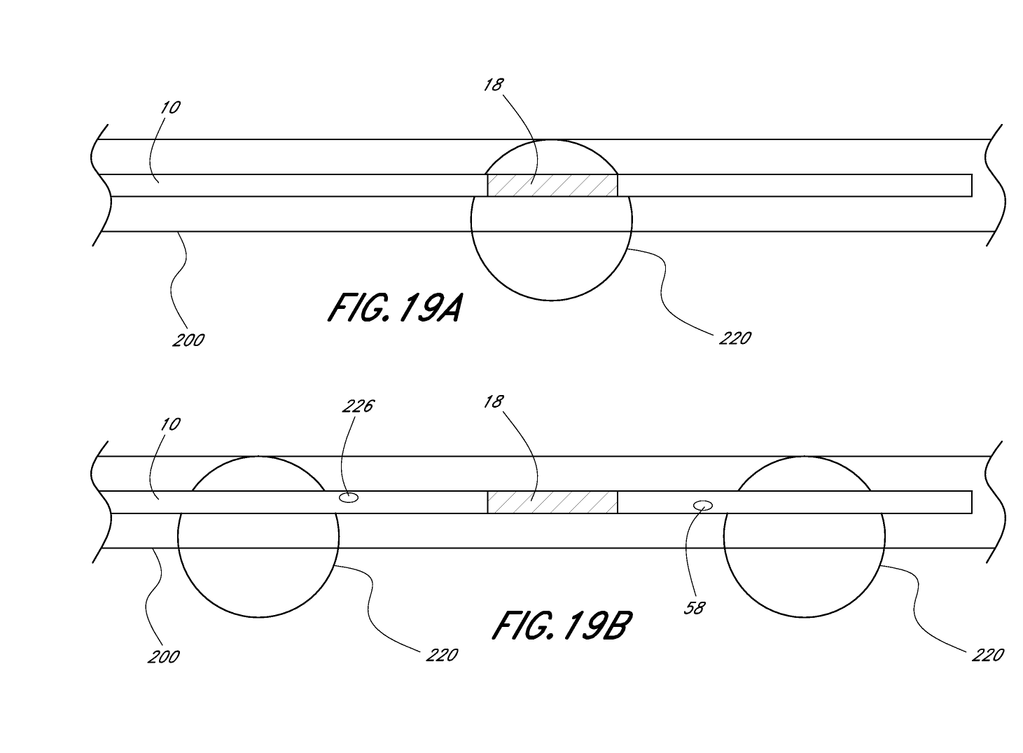

As illustrated in FIG. 18, a marking 224, such as a colored stripe or other visible, external ergonomic feature, can correspond to arc θ or indicate the center of arc θ, and indicate to the operator the direction of ultrasonic energy field and the correct orientation of the energy deliver section 18 of the ultrasound catheter 10. This marking 224 or feature can be located on a portion of the catheter 10 that remains outside the patient and is visible to the operator after the catheter 10 is positioned within the patient. This method of orienting the catheter 10 will be effective so long as the portion of the catheter 10 between the marker 224 or feature and the ultrasound energy delivery portion 18 does not substantially twist. The ultrasound catheter 10 can be configured to resist twisting but permit bending.

Alternatively, in some embodiments, an external ultrasound detector can be placed on the patient's chest over the heart. When the catheter 10 is being introduced it can put out a directional signal. The operator can rotate and translate the catheter 10 until the highest output is captured by the external detector, indicated correct targeting. Then the therapeutic power level can be delivered. In other embodiments, the catheter 10 can contain the ultrasound detector and an external ultrasound transducer can provide the signal source. The external ultrasound transducer can be placed on the patient's chest over the heart and the operator can rotate and translate the catheter 10 until the highest output is captured by the detector on the catheter 10. In other embodiments, the ultrasound transducers in the catheter 10 can be used to send out a signal, and a detector in the catheter 10 can monitor reflections of the signal off the patient's tissues and organs until the returning signal indicates heart muscle and not lung filled with air. In other embodiments, a therapeutic ultrasound catheter 10 can be attached to existing TEE probes, and the TEE probe can be used to target the heart.

The ultrasound catheter 10 can comprise a scanning array of ultrasound transducers so that the ultrasonic energy field can be targeted without moving the catheter. In addition, the array can be focused so that intensity at the heart is higher than in the esophagus. Furthermore, the array can be directed to apply a moving pressure gradient from the top of the vessel to its bottom, helping to force lytic drug into the clot.

For axial placement, the distance to the heart can be determined by measuring the distance from the sternal notch to the mouth or nasal cavity. In some embodiments, the catheter 10 can have a plurality of markers 224, where each marker corresponds to a particular sternal notch to mouth or nasal cavity distance, as illustrated in FIG. 18. To determine the correct distance in which to insert the catheter 10, the operator measures the sternal notch to mouth or nasal cavity distance, and then inserts the catheter 10 until the appropriate marker on the catheter shaft aligns with the mouth, teeth or the nasal openings.

Alternatively, the marker 224 or feature can indicate the appropriate distance in which to insert the catheter 10 such that the ultrasonic energy delivery portion 18 is aligned with the patient's heart by correlation with the patient's height. For a patient with a given height, the marker 224 would allow the operator to correctly orient the catheter 10 to direct the ultrasonic energy field to the heart. The catheter 10 can have multiple markers 224, corresponding to patients having a range of different heights, so that a single catheter 10 can be used for patients of different heights. In some embodiments, each marker 224 can specify the height of the patient to which it corresponds. For example, the most distal marker 224 can correspond to a patient having a height of three feet while the most proximal marker 224 can correspond to a patient having a height of 8 feet. In other embodiments, the most distal marker 224 can correspond to a height of about 4 feet or about 5 feet. In other embodiments, the most proximal marker 224 can correspond to a height of about 7 feet. To determine the correct distance in which to insert the catheter 10, the operator can determine the patient's height and then insert the catheter 10 until the appropriate marker 224 on the catheter shaft aligns with the mouth, teeth or the nasal openings.

Alternatively the tip may be imaged by external imaging equipment. The tip can be fabricated with a radiopaque marker that can be imaged using, for example, ultrasound or x-ray imaging equipment that can also image the heart.

In some embodiments, the power or intensity of the ultrasound energy delivered by the catheter 10 can be adjusted according to the patient's weight. For a larger patient, more power or ultrasound energy can be delivered relative to a smaller patient.

Because air transmits ultrasound energy relatively poorly, acoustic coupling of the catheter 10 to the esophagus 200 is desirable. Acoustic coupling of the catheter 10 to the esophagus 200 can be accomplished by inflating a balloon 220 around the ultrasound transducers in energy delivery section 18 with ultrasound gel or water until the balloon contacts the esophagus and forms a seal, as illustrated in FIG. 19A. In some embodiments, as shown in FIG. 19A, the balloon 220 can be mounted eccentrically so that the catheter is pushed to the heart side of the esophagus 200. Alternatively, in other embodiments two balloons 220 can be mounted proximal and distal to the energy delivery section 18 and the captive space can be filled with acoustic gel through a fluid delivery port 58 located in the catheter sheath and between the two balloons 220 after the balloons 220 are inflated. These two balloons 220 can also be mounted eccentrically so that the catheter 10 is pushed to the heart side of the esophagus 200. Air can be removed from the captive space as the acoustic gel is introduced by aspiration ports 226 or by permitting a gap between one of the balloons 200 and the esophagus wall during the filling period.

In some embodiments, the catheter 10 can be left in place for a period of time. A means for anchoring the catheter 10 relative to the patient can be provided to keep the catheter 10 properly positioned. The catheter 10 can be, for example, affixed, clamped, bound, taped, secured, or tied to an external positioning structure that can be located above the patient's head near the catheter's point of entry via the mouth or nasal cavity. Alternatively, the catheter 10 can be, for example, affixed, clamped, bound, taped, secured, or tied to the patient.

### SCOPE OF THE INVENTION

While the foregoing detailed description discloses several embodiments of the present invention, it should be understood that this disclosure is illustrative only and is not limiting of the present invention. It should be appreciated that the specific configurations and operations disclosed can differ from those described above, and that the methods described herein can be used in contexts other than treatment of vascular occlusions.

What is claimed is:

1. A method for treating a patient having an acute myocardial infarction, the method comprising:
providing an ultrasound catheter, the ultrasound catheter comprising an elongate body having a proximal portion and a distal portion, the distal portion comprising an ultrasound energy delivery section;
inserting the distal portion of the ultrasound catheter into the patient's esophagus;
generating an ultrasonic energy field that encompasses at least a portion of the patient's heart and coronary vasculature; and
introducing a thrombolytic drug to the patient intravenously.

2. The method of claim 1, wherein the ultrasound catheter further comprises a balloon mounted over the energy delivery section.

3. The method of claim 2, wherein the balloon is mounted eccentrically over the energy delivery section.

4. The method of claim 2, further comprising inflating the balloon with an acoustic gel or water until the balloon forms a seal around the patient's esophagus.

5. The method of claim 1, wherein the proximal portion comprises markings, the markings indicating the proper axial and rotational orientation of the ultrasound catheter within the patient's esophagus.

6. The method of claim 5, further comprising inserting the ultrasound catheter into the patient's esophagus via the patient's mouth until the markings are aligned with the patient's mouth.

7. The method of claim 6, further comprising applying a local anesthetic lubricant to the patient's mouth and throat.

8. The method of claim 5, further comprising inserting the ultrasound catheter into the patient's esophagus via the patient's nose until the markings are aligned with the patient's nose.

9. The method of claim 8, further comprising applying a local anesthetic lubricant to the patient's nasal cavity and throat.

10. The method of claim 1, further comprising aspirating fluids in the esophagus.

11. The method of claim 10, wherein the ultrasound catheter further comprises an aspiration port located in the distal portion.

12. An ultrasound catheter for treating a patient having an acute myocardial infarction, the ultrasound catheter comprising:
an elongate body, the elongate body having a proximal portion and a distal portion and a diameter less than the patient's esophagus;
an ultrasound energy delivery section located in the distal portion;
at least one marking on the proximal portion, the marking indicating the proper axial and rotational orientation of the ultrasound catheter within the patient's esophagus; and
a balloon mounted over the energy delivery section.

13. The ultrasound catheter of claim 12, wherein the balloon is mounted eccentrically over the energy delivery section.

14. The ultrasound catheter of claim 12, further comprising an aspiration port located on the distal portion.

15. The ultrasound catheter of claim 12, further comprising a fluid delivery port located on the distal portion.

16. The ultrasound catheter of claim 12, further comprising a cavity formed within the energy delivery section, wherein the cavity transmits ultrasound energy poorly.

17. The ultrasound catheter of claim 16, wherein the cavity is filled with air.

18. The ultrasound catheter of claim 17, wherein the cavity is a vacuum.
